(19) United States
(12) Patent Application Publication
(10) Pub. No.: US 2011/0318798 A1
(43) Pub. Date: Dec. 29, 2011

(54) REMOVAL OF INHIBITORS OF MICROBIAL FERMENTATION FROM INHIBITOR-CONTAINING COMPOSITIONS

(75) Inventors: David C. Walther, Oakland, CA (US); Eamon T. Hogg, Birmingham, AL (US); Gregory W. Luli, San Diego, CA (US); Robert Eckert, Auburn, WA (US); Jeffrey D. Keating, Sunnyvale, CA (US)

(73) Assignee: Cobalt Technologies, Inc., Mountain View, CA (US)

(21) Appl. No.: 13/168,674

(22) Filed: Jun. 24, 2011

Related U.S. Application Data
(60) Provisional application No. 61/358,353, filed on Jun. 24, 2010.

(51) Int. Cl. C12P 7/16; C12N 1/20

(52) U.S. Cl. 435/160; 435/253.6

(57) ABSTRACT

Methods are provided for conditioning an inhibitor-containing composition, such as a cellulosic biomass hydrolysate, to remove inhibitors of microbial growth and/or product production. The methods include precipitation of inhibitors by formation of complexes with metal salts, such as aluminum sulfate and ferric chloride.

### CROSS-REFERENCE TO RELATED APPLICATIONS

This application claims the benefit of U.S. Provisional Application No. 61/358,353, filed on Jun. 24, 2010, which is incorporated by reference herein in its entirety.

### FIELD OF THE INVENTION

The invention relates to a method for removing inhibitors of microbial growth and/or product production from an inhibitor-containing composition such as a hydrolysate of cellulosic biomass, in particular by forming a complex with a metal salt.

### BACKGROUND OF THE INVENTION

Many useful products may be produced by microorganisms grown in culture. A carbon source for such cultures is often provided by hydrolysis of cellulosic biomass materials. Soluble sugar molecules released by hydrolysis may be used to support microbial growth.

Hydrolysates of cellulosic materials often contain inhibitors of microbial growth and/or product production, which reduces the amount of product produced in a culture that includes such a hydrolysate. A number of methods have been developed for “conditioning” of hydrolysates to remove inhibitors prior to addition of the hydrolyzed material to microbial growth medium. Examples of conditioning processes that have been previously employed include vacuum or thermal evaporation, overliming, adsorption, enzymatic conditioning (e.g., peroxidase, laccase), chemical conversion, distillation, and ion exchange.

Overliming, i.e., calcium hydroxide precipitation, of hydrolysates to remove inhibitors is not effective at room temperature. The hydrolysate must be heated, which increases the cost of the process. Further, the calcium hydroxide—inhibitor complexes are difficult to remove and the overall process results in high sugar loss. New conditioning procedures that are effective at low temperature with minimal sugar loss are needed.

### BRIEF SUMMARY OF THE INVENTION

Methods for conditioning a composition that contains at least one inhibitor of microbial growth and/or bioproduct production and at least one compound suitable for use as a carbon source, such as a hydrolysate of a cellulosic material, are provided. Conditioned compositions, such as conditioned hydrolysates, prepared by the methods described herein are also provided, as well as methods for producing a bioproduct in a fermenting microorganism using the conditioned compositions described herein as a carbon source.

In one aspect, a method is provided for removing at least a portion of at least one (i.e., one or more) inhibitor of microbial growth and/or microbial bioproduct production from a composition that contains at least one inhibitor and at least one compound suitable for use as a carbon source in a microbial fermentation process, such as, for example, a hydrolysate of cellulosic biomass that contains inhibitor(s) and sugar molecule(s). The method includes contacting the composition with a metal salt. The metal salt forms a complex with the inhibitor(s) in the composition. The metal salt-inhibitor complex(es) is(are) separated from the liquid composition, thereby forming a conditioned composition.

In one aspect, a method is provided for reducing or eliminating the effect of an inhibitor of microbial growth and/or microbial bioproduct production in a composition that contains at least one inhibitor and at least one compound suitable for use as a carbon source in a microbial fermentation process, such as, for example, a hydrolysate of cellulosic biomass that contains inhibitor(s) and sugar molecule(s). The method includes contacting the composition with a metal salt. The metal salt forms a complex with the inhibitor(s) in the composition. In some aspects the effect of the inhibitor is reduced at least about 50%, 60%, 70%, 80% or 90%. In some aspects the effect of the inhibitor is reduced enough to measure a significant difference between production with and without the addition of a metal salt. In some aspects the effects which are reduced or eliminated are (i) adverse effects on microbial growth, or (ii) adverse effects on bioproduct production (for example adverse effects on titer, yield, and/or productivity of a product).

In one embodiment, the metal salt contains a metal ion with a valence of three or more, for example, a trivalent metal ion. In one embodiment, the metal salt contains a divalent metal ion, with the proviso that the divalent metal is not calcium. The metal salt may contain, for example, aluminum, iron, or magnesium, or a combination thereof. In one embodiment, the metal salt is a sulfate or halogen salt. In one embodiment, the metal salt contains aluminum, iron, or a combination thereof. In some embodiments, the metal salt is Al₂(SO4)₃ or FeCl₃. In one embodiment, the metal salt does not contain calcium. In one embodiment, the metal salt does not contain an alkaline earth metal. In some embodiments, the method is performed at a pH of about 7 to about 11, about 9.5 to about 11, or about 9.5 to about 10. In one embodiment, the pH is adjusted with a base, for example, ammonium hydroxide. In another embodiment, the pH is adjusted with ammonia gas. In some embodiments, the method is performed at a temperature of about 20° C. to about 60° C.

Typically, the metal salt is added to the composition at a concentration of about 1 t/L to about 6 g/L, about 3 g/L to about 5g/L, about 2 g/L to about 3 g/L, or about 2 g/L to about 4 g/L. In some embodiments, the metal salt is added at a concentration of any of about 1, 1.5, 2, 2.5, 3, 3.5, 4, 4.5, 5, 5.5, or 6 g/L.

In one embodiment, the composition comprises or consists of a hydrolysate of cellulosic biomass. In some embodiments, the cellulosic biomass is a lignocellulosic biomass, for example, softwood or hardwood, or a combination thereof. In one embodiment, the lignocellulosic biomass contains beetle killed Lodgepole pine. In some embodiments, the cellulosic biomass contains grass or straw, or a combination thereof. Nonlimiting examples of grass include sugar cane, miscanthus, switchgrass, or a combination thereof. Nonlimiting examples of straw include wheat straw, barley straw, rice straw, or a combination thereof. In some embodiments, the cellulosic biomass contains bagasse, cane trash, seaweed, algae, microalgae, agricultural waste or residue, hyacinth, sorghum, sugar beets, soybean residue, palm oil residue, or pulp mill liquor or effluent, or a combination thereof.

In some embodiments, a hydrolysate is produced by acid hydrolysis of cellulosic biomass, for example, with nitric acid, formic acid, acetic acid, phosphoric acid, hydrochloric acid, sulfuric acid, or a combination thereof. In other embodiments, a hydrolysate is produced by hot water extraction of cellulosic biomass, optionally followed by addition of acid (for example, nitric acid, formic acid acetic acid phosphoric acid hydrochloric acid or sulfuric acid, or a combination thereof) to further hydrolyze carbohydrate polymers.

In some embodiments, the composition comprises or consists of glycerol. In some embodiments, the composition comprises or consists of dairy effluent, for example, whey which contains lactose. In some embodiments, the composition comprises or consists of food waste, or a hydrolysate, e.g., an acid hydrolysate, of food waste.

In some embodiments, metal salt-inhibitor complex(es) is(are) separated from the liquid composition, e.g., cellulosic hydrolysate, by filtration, centrifugation, pressing, and/or decantation.

In some embodiments, the inhibitor(s) is(are) selected from organic acids (e.g., acetic, formic, levulinic), aldehydes (e.g., furfural, 5-hydroxymethyl furfural, vanillin), lignins, lignin byproducts or derivatives, inorganic salts (e.g., sulfates, phosphates, hydroxides), fatty acids, fatty alcohols, fats, waxes, polyesters (e.g., suberin), terpenoids, alkanes, wood extractives, Hibbert's ketones, and proteins. In one embodiment, the inhibitor(s) includes formic acid. In some embodiments, formic acid is reduced to a level that is not toxic to a bioproduct producing microbial species, for example, a solventogenic microbial species, such as a Clostridium species. In one embodiment, growth of a microorganism and/or bioproduct production in the microorganism is higher in the presence of a composition from which formic acid concentration has been reduced or removed in a conditioning method as described herein in comparison to a growth and/or bioproduct production in the presence of an identical composition from which has not been conditioned according to a method as described herein.

In some embodiments, the composition contains sugar molecules and no more than about 5% to about 10%, or about 10% to about 15%, for example, less than about 5%, 6%, 7%, 8%, 9%, 10%, 11%, 12%, 13%, 14%, or 15% of the sugar molecules are degraded in the conditioning methods described herein.

In another aspect, a conditioned composition, such as a conditioned hydrolysate, produced by any of the methods described herein, is provided. In some embodiments, the conditioned hydrolysate contains a level of formic acid that is not toxic to a bioproduct producing microbial species, for example, a solventogenic microbial species, such as a Clostridium species.

In another aspect, a method is provided for producing a bioproduct. The method includes culturing a microorganism in a medium that contains a conditioned composition, such as a conditioned hydrolysate, produced by any of the methods described herein. In one embodiment, growth of and/or bioproduct production in the microorganism is greater in the medium that contains the conditioned composition than in an otherwise identical medium that does not contain the conditioned composition, i.e., a medium that is otherwise identical but contains the starting composition from which the conditioned composition was derived in place of the conditioned composition. In one embodiment, the bioproduct is produced in a greater amount, at a greater yield, and/or at a greater rate than in an otherwise identical medium that does not contain the conditioned composition. In some embodiments, the bioproduct is produced at a greater titer, yield, and/or productivity than in an otherwise identical medium that does not contain the conditioned composition.

In one embodiment, the bioproduct is a solvent. In one embodiment, the bioproduct is a biofuel, for example, butanol, ethanol, acetone, or a combination thereof. In one embodiment, the bioproduct is a biochemical or biochemical intermediate, for example, formate, acetate, butyrate, propionate, succinate, methanol, propanol, or hexanol.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The invention provides methods for removal of inhibitors of microbial growth and/or product production from compositions that contain such inhibitors and compounds that are suitable for use as a carbon source for microbial fermentation, such as hydrolysates of cellulosic material. The methods provided herein include formation of complexes between the inhibitors and one or more metal salts. Methods are also provided for using the conditioned compositions from which inhibitors have been removed to support microbial growth for bioproduct production.

Advantageously, the conditioning methods herein may be performed at relatively low temperature with minimal sugar loss, and the metal salt complexes are easy to separate from the liquid composition, with the removal of the complexes achievable with standard laboratory equipment.

Unless defined otherwise herein, all technical and scientific terms used herein have the same meaning as commonly understood by one of ordinary skill in the art to which this invention belongs. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present invention.

The practice of the present invention will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); and Gene Transfer and Expression: A Laboratory Manual (Kriegler, 1990).

Numeric ranges provided herein are inclusive of the numbers defining the range.

Unless otherwise indicated, nucleic acids are written left to right in 5′ to 3′ orientation; amino acid sequences are written left to right in amino to carboxy orientation, respectively.

### Definitions

“A,” “an” and “the” include plural references unless the context clearly dictates otherwise.

“Bioproduct” refers to any substance of interest produced biologically, i.e., via a metabolic pathway, by a microorganism, e.g., in a microbial fermentation process. Bioproducts include, but are not limited to biofuels (e.g., butanol, acetone, ethanol), solvents, biomolecules (e.g., proteins (e.g., enzymes), polysaccharides), organic acids (e.g., formate, acetate, butyrate, propionate, succinate), alcohols (e.g., methanol, propanol, isopropanol, hexanol, 2-butanol, isobutanol), fatty acids, aldehydes, lipids, long chain organic molecules (for example, for use in surfactant production), vitamins, and sugar alcohols (e.g., xylitol).

“Biofuel” refers to fuel molecules (e.g., butanol, acetone, and/or ethanol) produced biologically by a microorganism, e.g., in a microbial fermentation process.

“Biobutanol” refers to butanol (i.e., n-butanol) produced biologically by a microorganism, e.g., in a microbial fermentation process.

“Byproduct” refers to a substance that is produced and/or purified and/or isolated during any of the processes described herein, which may have economic or environmental value, but that is not the primary process objective. Nonlimiting examples of byproducts of the processes described herein include lignin compounds and derivatives, carbohydrates and carbohydrate degradation products (e.g., furfural, hydroxymethyl furfural, formic acid), and extractives (described infra).

“Feedstock” refers to a substance that can serve as a source of sugar molecules to support microbial growth in a fermentation process. In some embodiments, the feedstock must be pretreated to release the sugar molecules. In one embodiment, the feedstock, which contains carbohydrate polymers, is hydrolyzed to release 5 and/or 6 carbon containing carbohydrate molecules in monomeric and/or soluble oligomeric forms.

“Deconstruction” refers to mechanical, chemical, and/or biological degradation of biomass into to render individual components (e.g., cellulose, hemicellulose) more accessible to further pretreatment processes, for example, a process to release monomeric and oligomeric sugar molecules, such as acid hydrolysis.

“Conditioning” refers to removal of inhibitors of microbial growth and/or bioproduct, e.g., biofuel, production from a hydrolysate produced by hydrolysis of a cellulosic feedstock.

“Titer” refers to amount of a substance produced by a microorganism per unit volume in a microbial fermentation process. For example, biobutanol titer may be expressed as grams of butanol produced per liter of solution.

“Yield” refers to amount of a product produced from a feed material (for example, sugar) relative to the total amount that of the substance that would be produced if all of the feed substance were converted to product. For example, biobutanol yield may be expressed as % of biobutanol produced relative to a theoretical yield if 100% of the feed substance (for example, sugar) were converted to biobutanol.

“Productivity” refers to the amount of a substance produced by a microorganism per unit volume per unit time in a microbial fermentation process. For example, biobutanol productivity may be expressed as grams of butanol produced per liter of solution per hour.

“Wild-type” refers to a microorganism as it occurs in nature.

“Biomass” refers to cellulose- and/or starch-containing raw materials, including but not limited to wood chips, corn stover, rice, grasses, forages, perrie-grass, potatoes, tubers, roots, whole ground corn, grape pomace, cobs, grains, wheat, barley, rye, milo, brans, cereals, sugar-containing raw materials (e.g., molasses, fruit materials, sugar cane, or sugar beets), wood, and plant residues.

“Starch” refers to any starch-containing materials. In particular, the term refers to various plant-based materials, including but not limited to wheat, barley, potato, sweet potato, tapioca, corn, maize, cassava, milo, rye, and brans. In general, the term refers to any material comprised of the complex polysaccharide carbohydrates of plants, comprised of amylose, and amylopectin, with the formula (C₆H₁₀O₅)ₓ, wherein “x” can be any number.

“ABE fermentation” refers to production of acetone, butanol, and/or ethanol by a fermenting microorganism.

“Advanced biofuels” are high-energy liquid transportation fuels derived from low nutrient input/high per acre yield crops, agricultural or forestry waste, or other sustainable biomass feedstocks including algae.

“Lignocellulosic” biomass refers to plant biomass that contains cellulose, hemicelluloses, and lignin. The carbohydrate polymers (cellulose and hemicelluloses) are tightly bound to lignin.

“Lignins” are macromolecular components of wood that contain phenolic propylbenzene skeletal units linked at various sites.

“Solvent” refers to a liquid or gas produced by a microorganism that is capable of dissolving a solid or another liquid or gas. Nonlimiting examples of solvents produced by microorganisms include n-butanol, acetone, ethanol, acetic acid, isopropanol, n-propanol, methanol, formic acid, 1,4-dioxane, tetrahydrofuran, acetonitrile, dimethylformamide, and dimethyl sulfoxide.

A “protic” solvent contains dissociable H⁺, for example a hydrogen atom bound to an oxygen atom as in a hydroxyl group or a nitrogen atom as in an amino group. A protic solvent is capable of donating a proton (H⁺). Conversely, an “aprotic” solvent cannot donate H⁺.

n-Butanol is also referred to as “butanol” herein.

“ATCC” refers to the American Type Culture Collection, P.O. Box 1549, Manassas, Va. 20108.

### Methods for Conditioning an Inhibitor-Containing Composition

A method is provided for conditioning of a composition that contains at least one inhibitor of microbial growth and/or bioproduct production and at least one compound that may serve as a carbon source for microorganism growth. In one embodiment, the carbon source contains sugar molecules, e.g., monosaccharides. In another embodiment, the carbon source contains glycerol. Conditioning of the composition, i.e., removal of at least a portion of at least one inhibitor, improves its ability to support growth of a microorganism and/or bioproduct production in a microbial fermentation. The composition is conditioned by adding a metal salt for a period of time and under conditions, e.g., temperature, pH, such that at least a portion of at least one inhibitor forms a complex with the metal salt, which is then separated and removed from the composition. In one embodiment, substantially all of at least one inhibitor is removed.

The metal salt may contain a metal with a valence of three of more (i.e., a trivalent metal or metal with a higher valence than three) or may contain a divalent metal, with the proviso that the divalent metal is not calcium. In some embodiments, the metal salt contains aluminum, iron or magnesium, or a combination thereof.

In one embodiment, the metal salt contains a trivalent metal ion, for example, aluminum or iron. In one embodiment, the metal salt does not contain calcium. In one embodiment, the metal salt does not contain an alkaline earth metal. In one embodiment, the metal salt contains aluminum or iron. In one embodiment, the metal salt is Al₂(SO₄)₃ or FeCl₃, or a combination thereof.

In one embodiment, hydrolyzed cellulosic feedstock, which may be prepared as described infra, is “conditioned” to remove inhibitors of microbial growth and/or bioproduct production, prior to addition of the hydrolyzed feedstock to a microbial growth medium. Such inhibitors may include, but are not limited to, organic acids, furans, phenols, soluble lignocellulosic materials, extractives, and ketones. Inhibitors present in wood hydrolysates may include, but are not limited to, 5-hydroxyy-methyl furfural (HMF), furfural, aliphatic acids, levulinic acid, acetic acid, formic acid, phenolic compounds, vanillin, dihydroconiferylalcohol, coniferyl aldehyde, vanillic acid, hydroquinone, catechol, acetoguaiacone, homovanillic acid, 4-hydroxy-benzoic acid, Hibbert's ketones , ammonium nitrate, p-coumaric acid, ferulic acid, 4-hydroxybenzoic acid, vanillic acid, syringaldehyde, and glucuronic acid.

The hydrolysate is conditioned by adding one or more metal salt, which forms a complex with inhibitor(s) present in the hydrolysate. The complex is separated from the liquid hydrolysate, thereby producing a conditioned hydrolysate in which the inhibitor(s) are depleted or absent.

In a multiple stage hydrolysis process, as described infra, hydrolysate produced in the first stage and optionally in subsequent stage(s) may be conditioned as described herein.

In some embodiments, the method is performed with the pH of the hydrolysate adjusted to about 7 to about 11, about 9.5 to about 11, or about 9.5 to about 10. In some embodiments, the pH is adjusted to any of about 9.5, 10, 10.5, or 11, or any of about 9 to about 9.5, about 9.5 to about 10, about 10 to about 10.5, or about 10.5 to about 11. In one embodiment, the pH of the, acidic hydrolysate solution is adjusted with a base for example, ammonium hydroxide. In one embodiment, the pH is adjusted with ammonia gas. In some embodiments, the method is performed at a temperature of about 20° C. to about 60° C. In some embodiments, the method is performed at a temperature of any of about 20° C., 25° C., 30° C., 35° C., 40° C., 45° C., 50° C., 55° C., or 60° C. In some embodiments, the method is performed at any of about 20° C. to about 25° C., about 25° C. to about 30° C., about 30° C. to about 35° C., about 35° C. to about 40° C., about 40° C. to about 45° C., about 45° C. to about 50° C., about 50° C. to about 55° C., or about 55° C. to about 60° C.

The metal salt is added to the hydrolysate in an amount sufficient to form a separable complex with a substantial amount of the inhibitor compounds present in the hydrolysate. Typically, the metal salt is added to the hydrolysate at a concentration of about 1 g/L about 6 g/L, about 3 g/L to about 5 g/L, about 2 g/L to about 3 g/L, or about 2 g/L to about 4 g/L. In some embodiments, the metal salt is added at a concentration of any of about 1, 1.5, 2, 2.5, 3, 3.5, 4, 4.5, 5, 5.5, or 6 g/L.

Separation of the metal salt-inhibitor complexes may be performed by any technique by which liquids and solids may be separated, including by not limited to, filtration, impingement, pressing, passive settling (e.g., lamella separator, settling tank), active settling (e.g., centrifugation, hydrocyclone), or decantation.

At least a portion of at least one inhibitor (i.e., at least a portion of one inhibitor or at least a portion of each of more than one inhibitor) of microbial growth and/or bioproduct production is removed from an inhibitor-containing composition by treatment with a metal salt in a method as described herein. In some embodiments, at least about 50, 60, 70, 80, 90, 95, 98, or 99% of at least one inhibitor is removed. In some embodiments, at least a portion of each of a multiplicity of inhibitors (i.e., two or more inhibitors) is removed. In some embodiments, at least about 50, 60, 70, 80, 90, 95, 98, or 99% of each of a multiplicity of inhibitors is removed. In some embodiments, substantially all of at least one inhibitor or substantially all of each of a multiplicity of inhibitors is removed.

### Conditioned Compositions

A conditioned composition, from which at least a portion of at least one inhibitor of microbial growth and/or bioproduct production has been removed by a method as described herein, is provided. A conditioned composition herein, from which at least a portion or substantially all of at least one inhibitor has been removed by precipitation by a metal salt as described herein, contains at least one carbon source for microbial fermentation. The conditioned composition may be added to a growth medium or may be used as a growth medium for fermentation of a microorganism, for example, for production of one or more bioproduct(s).

In some embodiments, conditioned cellulosic hydrolysates are provided, prepared by any of the methods described herein. Formation of metal salt complexes with inhibitors of microbial growth and/or product production, and removal of the complexes, from a hydrolysate of a cellulosic material, provides a conditioned hydrolysate. Such a conditioned hydrolysate, which contains soluble carbohydrate molecules, may be used as a carbon source in a microbial fermentation to produce one or more bioproduct(s) of interest.

A conditioned hydrolysate may be prepared by hydrolysis of any of the cellulosic biomass materials described herein, followed by conditioning with any of the metal salts and under any of the process conditions described herein. In some embodiments, the cellulosic biomass material is a lignocellulosic biomass material.

### Feedstock

A feedstock is a substance that provides the base material from which sugar molecules are generated for inclusion in a microbial growth medium, to support the growth of the microorganism. Feedstock used in the methods described herein may be cellulosic biomass, for example, lignocellulosic biomass.

Cellulose, which is a β-glucan built up of D-glucose units linked by β(1,4)-glycosidic bonds, is the main structural component of plant cell walls and typically constitutes about 35-60% by weight (% w/w) of lignocellulosic materials.

Hemicellulose refers to non-cellulosic polysaccharides associated with cellulose in plant tissues. Hemicellulose frequently constitutes about 20-35% w/w of lignocellulosic materials, and the majority of hemicelluloses consist of polymers based on pentose (five-carbon) sugar units, such as D-xylose and D-arabinose units, hexose (six-carbon) sugar units, such as D-glucose and D-mannose units, and uronic acids such as D-glucuronic acid.

Lignin, which is a complex, cross-linked polymer based on variously substituted p-hydroxyphenylpropane units, typically constitutes about 10-30% w/w of lignocellulosic materials.

Any material containing cellulose may be used as the feedstock. The material may contain cellulose and hemicellulose with or without lignin.

In some embodiments, the feedstock is woody biomass. In one embodiment, the feedstock is softwood, for example, pine, e.g., Lodgepole or Loblolly pine. In one embodiment, the feedstock contains mountain pine beetle infested pine, for example, dying (“red stage”) or dead (“grey” stage). In another embodiment, the feedstock is hardwood, for example, maple, birch, or ash. In another embodiment, the feedstock is mixed hardwood and softwood. In another embodiment, the feedstock is mixed hardwood. In some embodiments, the woody biomass is in the form of wood chips, sawdust, saw mill residue, wood fines, or a combination thereof.

In some embodiments, the feedstock is obtained as a process stream from a biomass processing facility, for example, a pulp mill. In various embodiments of pulp mill process streams, the process stream may include reject pulp, wood knots or shiver, pulp screening room rejects (e.g., essentially cellulose in water), prehydrolysis extraction stream, and/or black liquor. Lignocellulose contains a mixture of carbohydrate polymers and non-carbohydrate compounds. The carbohydrate polymers contain cellulose and hemicellulose, and the non-carbohydrate portion contains lignin. The non-carbohydrate portion may also contain ash, extractives, and/or other components. The specific amounts of cellulose, hemicelluloses, and lignin depends on the source of the biomass. For example, municipal solid waste may contain primarily cellulose, and extract streams from a paper and pulp plant may contain primarily hemicelluloses. The remaining composition of lignocellulose may also contain other compounds such as proteins.

In some embodiments, the feedstock is a lignocellulosic material in the form of wood chips, sawdust, saw mill residue, or a combination thereof. In some embodiments, the lignocellulosic material (e.g., wood chips sawdust, saw mill residue, or a combination thereof) is from a feedstock source that has been subjected to some form of disease in the growth and/or harvest production period. In one embodiment, the feedstock source is mountain pine beetle infested pine. In another embodiment, the feedstock source is sudden oak death syndrome infested oak, e.g., coastal live oak, tanoak, etc. In another embodiment, the feedstock source is Dutch elm disease infested elm. In other embodiments, the feedstock source is lignocellulosic material that has been damaged by drought or fire.

Lignocellulosic biomass may be derived from a fibrous biological material such as wood or fibrous plants. Examples of suitable types of wood include, but are not limited to, spruce, pine, hemlock, fir, birch, aspen, maple, poplar, alder, salix, cottonwood, rubber tree, marantii, eucalyptus, sugi, and acase. Examples of suitable fibrous plants include, but are not limited to, corn stover and fiber, flax, hemp, cannabis, sisal hemp, bagasse, straw, cereal straws, reed, bamboo, miscanthus, kenaf, canary reed, Phalaris arundinacea, and grasses. Other lignocellulosic materials may be used such as herbaceous material, agricultural crop or plant residue, forestry residue, municipal solid waste, pulp or paper mill residue, waste paper, recycling paper, or construction debris. Examples of suitable plant residues include, but are not limited to, stems, leaves, hulls, husks, cobs, branches, bagasse, wood chips, wood pulp, wood pulp, and sawdust. Examples of suitable waste paper include, but are not limited to, discarded paper of any type (e.g., photocopy paper, computer printer paper, notebook paper, notepad paper, typewriter paper), newspaper, magazines, cardboard, and paper-based packaging material. Materials with high mineral content may potentially require additional pH adjustment (e.g., additional amounts of chemicals for pH adjustment) for effective processing.

Other feedstocks that may be used in the methods herein include hemicellulose extract from wood, beet extract, beet molasses, sorghum syrup, barley hulls, potato processing waste, and brewers mash.

In some embodiments, a feedstock mix containing about 40% logging residues, about 20% sustainable roundwood, about 20% woody energy crops, and about 20% herbaceous energy crops may be used. This blend can account for regional variation and provide significant flexibility in selecting locations for facilities and in procuring feedstock supply contracts.

In some embodiments, the feedstock contains grass, for example, sugar cane, miscanthus, and/or switchgrass, and/or straw, for example, wheat straw, barley straw, and/or rice straw.

### Pretreatment of Feedstock

Feedstocks such as those described herein can be pretreated using a variety of methods and systems prior to bioconversion. Preparation of the feedstock can include chemical or physical modification of the feedstock. For example, the feedstock can be shredded, sliced, chipped, chopped, heated, burned, dried, separated, extracted, hydrolyzed, and/or degraded. These modifications can be performed by biological, non-biological, chemical, or non-chemical processes.

In some embodiments in which a cellulosic, e.g., lignocellulosic, feedstock is used, processes may be used to break down cellulose and/or hemicellulose into sugar molecules that may be more easily processed by a microorganism. Processes that may be used include acid hydrolysis, enzymatic hydrolysis, gasification, pyrolysis, and cellulose degradation by a microorganism.

### Deconstruction

In some embodiments, the feedstock, such as lignocellulosic feedstock, for example, wood chips, sawdust, and/or sawdust residue, is deconstructed prior to a downstream pretreatment process such as hydrolysis. Deconstruction may include, but is not limited to, presteaming to swell and loosen material, mechanical grinding, mechanical explosion (e.g., steam or other chemical treatment followed by rapid decompression), vacuum treatment, acid-feedstock contact (diffusion of acid into feedstock), or a combination thereof. In some embodiments, deconstruction renders cellulose and/or hemicellulose in the feedstock more accessible for hydrolysis.

### Removal of Extractives

In some embodiments, the feedstock, such as lignocellulosic feedstock, for example, wood chips, sawdust, and/or sawdust residue, is pretreated to remove extractives. Extractives are material that is extracted from the feedstock by a process such as compression, water or solvent extraction, or air drying. Non-limiting examples of extractives include terpenes, resin acids, fatty acids, sterols, steryl esters, phenolic compounds, and triglycerides. Extractives may include, but are not limited to, p-coumaric acid, ferulic acid, 4-hydroxybenzoic acid, vanillic acid, syringaldehyde, vanillin, furfural, hydroxymethylfurfural, and glucuronic acid. Extractives may be removed for other uses, such as production of sterols, or burned to provide energy for a bioproduct, e.g., biofuel, production process as described herein.

In some embodiments, extractives are removed prior to or in conjunction with deconstruction of the feedstock.

### Hydrolysis of Cellulosic Biomass

Typically, a feedstock contains sugar molecules in an oligomeric form, e.g., a polymeric form, and must be hydrolyzed to extract and release soluble monomeric and/or multimeric sugar molecules, which are converted to bioproduct, e.g., biofuel, in a microbial fermentation as described herein. The sugar molecules are present in the feedstock in cellulose and optionally also in hemicellulose. In one embodiment, the feedstock is lignocellulosic biomass and the sugar molecules are present in the feedstock in cellulose and hemicellulose.

In some embodiments, the feedstock is pretreated with an acid hydrolysis process. Acids that may be used for hydrolysis include, but are not limited to, nitric acid, formic acid, acetic acid, phosphoric acid, hydrochloric acid, and sulfuric acid, or a combination thereof. In one embodiment, acid hydrolysis is performed in a single stage. In some embodiments, acid hydrolysis is performed in two or more stages, under different conditions in each stage to hydrolyze different components of the feedstock in each stage. In one embodiment, the second stage hydrolysis is performed at a higher temperature than the first stage hydrolysis. Acid hydrolysis performed in multiple stages may serve to limit the impact of kinetically controlled carbohydrate degradation mechanisms.

An acid hydrolysis system may be designed to submerge and flood the feedstock with the acid solution in the hydrolysis reactor, e.g., in a vertical section of the hydrolysis reactor, to insure even acid impregnation. Even heat distribution may be obtained by using both direct steam injection and a jacketed vessel in conjunction with a mechanical screw auger. Variable speed drives may be used with temperature sensing instrumentation to control reactor residence time and temperature allowing reactor severity to be adjusted on-line. Alternative reactor configurations with functionally similar properties may also be utilized. For example, a horizontal digestor configuration may be used. In this type of reactor, the material is only partially submerged. Similarly, in some embodiments, in order to reach higher soluble sugar concentrations, the feedstock material is not completely submerged in the acid containing solution, thereby producing a hydrolysate that contains an increased sugar concentration (i.e., less dilution water added at the outset).

In some embodiments, a multiple-stage dilute nitric acid hydrolysis process is used. In one embodiment, a two-stage dilute nitric acid process is used for hydrolysis of lignocellulosic feedstock. In one embodiment, conditions in the first stage are chosen to achieve hydrolysis of about 70% to about 90% of the hemicellulose in the feedstock and conditions in the second stage are chosen to achieve hydrolysis of about 40% to about 70% of the cellulose in the feedstock. The first stage mainly targets the hydrolysis of the hemicellulose, yielding a mannose and/or xylose rich hydrolysate, whereas the second stage uses the solids remaining from the first stage and targets the cellulose, yielding a glucose rich hydrolysate. Typically, first stage hydrolysate liquors contain a mix of 5-carbon and 6-carbon sugars, e.g., extracted primarily from hemicellulose and non-recalcitrant cellulose biomass components, and second stage hydrolysate contains primarily 6-carbon sugars, e.g., extracted from cellulose fibers, in both cases as soluble monomeric and/or multimeric forms. 6-carbon monosaccharides may include, but are not limited to, glucose, mannose, and galactose. 6-carbon disaccharides may include, but are not limited to, cellobiose, mannobiose, glucomannose, and galactomannose. Other multimeric forms may include, but are not limited to, cellotriose, cellotetrose, and cellopentose. 5-carbon monosaccharides may include, but are not limited to xylose and arabinose. 5-carbon disaccharides and other multimeric forms may include, but are not limited to, xylobiose, xylotriose, and arabinoxylose.

In some embodiments in which hardwood is used as the feedstock, the first stage hydrolysate contains about 60% to about 75% 5-carbon sugar by weight and about 25% to about 40% 6-carbon sugar by weight, and the second stage hydrolysate contains about 80% to about 95% 6-carbon sugar by weight. In some embodiments in which softwood is used as the feedstock, the first stage hydrolysate contains about 20% to about 30% 5-carbon sugar by weight and about 70% to about 80% 6-carbon sugar by weight, and the second stage hydrolysate contains about 90% to about 100% 6-carbon sugar by weight, wherein the second stage is performed at a higher temperature than the first stage.

A first stage hydrolysis module may be coupled to a second stage hydrolysis module, with solid residue separated from liquid hydrolysate generated in the first stage hydrolysis serving as substrate for the second hydrolysis process. The residual solids may be rinsed/washed in order to increase the separation and recovery yield of soluble sugars separated from the biomass.

In some embodiments of a two stage hydrolysis process, hydrolysis is performed at a nitric acid concentration of about 0.1% to about 0.5%, about 0.5% to about 1%, about 1% to about 4%, about 1.3% to about 3.5%, or about 1.3% (w/w of dry feedstock) for both hydrolysis stages, at a temperature of about 170° to about 175° C. in the first stage and a temperature of about 210° to about 230° C. in the second stage, and at the saturation pressure for steam at the reactor temperature for each hydrolysis stage.

In some embodiments, the liquid (acid) to solid (feedstock) ratio for hydrolysis is about 10:1 to about 5:1 or about 7.5:1 to about 5:1. In a circulating reactor, the ratio of liquid to solid may be about 5:1 to about 3:1 or about 3.5:1 to about 3:1. In a continuous extrusion reactor, the ratio of liquid to solid may be about 4:1 to about 0.5:1.

### Methods for Producing a Bioproduct

Methods are provided for producing a bioproduct. The methods include culturing a microorganism in a medium that contains a conditioned composition, e.g., a conditioned hydrolysate, prepared according to any of the methods described herein. The conditioned composition provides a carbon source, for example, soluble sugar molecules. In some embodiments, microbial growth and/or bioproduct titer, yield, and/or productivity is increased when a conditioned composition, for example, conditioned hydrolyzed feedstock, as described herein is used in a microbial fermentation process, in comparison to identical hydrolyzed feedstock which has not been subjected to the conditioning process.

In some embodiments, the bioproduct is a solvent, such as, for example, a polar aprotic or protic solvent. In some embodiments, the solvent is n-butanol, acetic acid, isopropanol, n-propanol, ethanol, methanol, formic acid, 1,4-dioxane, tetrahydrofuran, acetone, acetonitrile, dimethylformamide, or dimethyl sulfoxide, or a combination thereof.

In some embodiments, the bioproduct is a biofuel, for example, butanol, ethanol, or acetone, or a combination thereof.

In some embodiments, the bioproduct is a biochemical or biochemical intermediate, for example, formate, acetate, butyrate, propionate, succinate, methanol, propanol, or hexanol, or a combination thereof.

### Fermentation

The methods for bioproduct production herein include fermentation of a bioproduct-producing microorganism in a bioreactor in a growth medium that contains a conditioned composition, for example, a conditioned hydrolysate, prepared as described herein.

In some embodiments, the bioproduct production includes fermentation of a bioproduct-producing microorganism in an immobilized cell bioreactor (i.e., a bioreactor containing cells that are immobilized on a support, e.g., a solid support). In some embodiments, an immobilized cell bioreactor provides higher productivity due to the accumulation of increased productive cell mass within the bioreactor compared with a stirred tank (suspended cell) bioreactor. In some embodiments, the microbial cells form a biofilm on the support and/or between support particles in the growth medium.

In some embodiments, the bioproduct production process herein includes continuous fermentation of a microorganism (continuous addition of conditioned hydrolyzed feedstock and withdrawal of product stream). Continuous fermentation minimizes the unproductive portions of the fermentation cycle, such as lag, growth, and turnaround time, thereby reducing capital cost, and reduces the number of inoculation events, thus minimizing operational costs and risk associated with human and process error.

Fermentation may be aerobic or anaerobic, depending on the requirements of the bioproduct-producing microorganism.

In some embodiments, an immobilized bioproduct-producing Clostridium strain is fermented anaerobically in a continuous process as described herein.

One or more bioreactors may be used in the bioproduct production systems and processes described herein. When multiple bioreactors are used they can be arranged in series and/or in parallel. The advantages of multiple bioreactors over one large bioreactor include lower fabrication and installation costs, ease of scale-up production, and greater production flexibility. For example individual bioreactors may be taken off-line for maintenance, cleaning, sterilization, and the like without appreciably impacting the production schedule. In embodiments in which multiple bioreactors are used, the bioreactors may be run under the same or different conditions.

In a parallel bioreactor arrangement, hydrolyzed feedstock is fed into multiple bioreactors, and effluent from the bioreactors is removed. The effluent may be combined from multiple bioreactors for recovery of the bioproduct, or the effluent from each bioreactor may be collected separately and used for recovery of the bioproduct.

In a series bioreactor arrangement, hydrolyzed feedstock is fed into the first bioreactor in the series, the effluent from the first bioreactor is fed into a second downstream bioreactor, and the effluent from each bioreactor in the series is fed into the next subsequent bioreactor in the series. The effluent from the final bioreactor in the series is collected and may be used for recovery of the bioproduct.

Each bioreactor in a multiple bioreactor arrangement can have the same species, strain, or mix of species or strains of microorganisms or a different species, strain, or mix of species or strains of microorganisms compared to other bioreactors in the series.

In some embodiments, feedstock is hydrolyzed in a multi-stage process as described herein, conditioned as described herein, and conditioned composition, e.g., conditioned hydrolysate, from each stage is fed to a separate bioreactor. The bioreactors to which the different conditioned compositions are fed may contain the same or different microbial species or strains. In one embodiment, the bioreactors to which the different compositions are fed contain different microbial species or strains that have each been optimized for growth on the particular composition, e.g., hydrolysate, being fed to that bioreactor. In some embodiments, different sets of multiple bioreactors in series are fed hydrolysate from different stages of hydrolysis of a cellulosic feedstock.

Immobilized cell bioreactors allow higher concentrations of productive cell mass to accumulate and therefore, the bioreactors can be run at high dilution rates, resulting in a significant improvement in volumetric productivity relative to cultures of suspended cells. Since a high density, steady state culture can be maintained through continuous culturing, with the attendant removal of product containing fermentation broth, smaller capacity bioreactors can be used. Bioreactors for the continuous fermentation of C. acetobutylicum are known in the art. (U.S. Pat. Nos. 4,424,275, and 4,568,643.)

Numerous methods of fermentor inoculation are possible including addition of a liquid seed culture to the bottom or the top of the bioreactor and recirculation of the media to encourage growth throughout the bed. Other methods include the addition of a liquid seed culture or impregnated solid support through a port located along the reactor's wall or integrated and loaded with the solid support material. Bioreactor effluent may also be used to inoculate an additional bioreactor and in this case any residual fermentable materials may be converted in the secondary reactor, increasing yield/recovery.

In a similar manner, support material may be added to the reactor through bottom, top, or side loading to replenish support material that becomes degraded or lost from the bioreactor.

### Fermentation Media

Fermentation media for the production of bioproduct contain feedstock, e.g., a conditioned hydrolyzed feedstock, as described herein, as a source of fermentable carbohydrate molecules.

As known in the art, in addition to an appropriate carbon source, fermentation media must contain suitable nitrogen source(s), mineral salts, cofactors, buffers, and other components suitable for the growth of the cultures and promotion of the enzymatic pathway necessary for the production of the desired bioproduct. In some embodiments, salts and/or vitamin B12 or precursors thereof are included in the fermentation media. In some cases, hydrolyzed feedstock may contain some or all of the nutrients required for growth, minimizing or obviating the need for additional supplemental material.

The nitrogen source may be any suitable nitrogen source, including but not limited to, ammonium salts, yeast extract, corn steep liquor (CSL), and other protein sources including, but not limited to, denatured proteins recovered from distillation of fermentation broth or extracts derived from the residual separated microbial cell mass recovered after fermentation. Phosphorus may be present in the medium in the form of phosphate salts, such as sodium, potassium, or ammonium phosphates. Sulfur may be present in the medium in the form of sulfate salts, such as sodium or ammonium sulfates. Additional salts include, but are not limited to, magnesium sulfate, manganese sulfate, iron sulfate, magnesium chloride, calcium chloride, manganese chloride, ferric chloride, ferrous chloride, zinc chloride, cupric chloride, cobalt chloride, and sodium molybdate. The growth medium may also contain vitamins such as thiamine hydrochloride, biotin, and para-aminobenzoic acid (PABA). The growth medium may also contain one or more buffering agent(s) (e.g., MES), one or more reducing agent(s) (e.g., cysteine HCl), and/or sodium lactate, which may serve as a carbon source and pH buffer.

### Microorganisms

The systems and processes described herein include one or more microorganism(s) that is (are) capable of producing one or more bioproduct(s) of interest. In embodiments in which two or more microorganisms are used, the microorganisms may be the same or different microbial species and/or different strains of the same species.

In some embodiments, the microorganisms are bacteria or fungi. In some embodiments, the microorganisms are a single species. In some embodiments, the microorganisms are a mixed culture of strains from the same species. In some embodiments, the microorganisms are a mixed culture of different species. In some embodiments, the microorganisms are an environmental isolate or strain derived therefrom.

In some embodiments of the processes and systems described herein, different species or strains, or different combinations of two or more species or strains, are used in different bioreactors with different conditioned hydrolyzed feedstocks as a carbohydrate source.

In some embodiments, a fungal microorganism is used, such as a yeast. Examples of yeasts include, but are not limited to, Saccharomyces cerevisiae, S. bayanus, S. carlsbergensis, S. Monacensis, S. Pastorianus, S. uvarum and Kluyveromyces species. Other examples of anaerobic or aerotolerant fungi include, but are not limited to, the genera Neocallimastix, Caecomyces, Piromyces and other rumen derived anaerobic fungi.

In some embodiments, a bacterial microorganism is used, including Gram-negative and Gram-positive bacteria. Non-limiting examples of Gram-positive bacteria include bacteria found in the genera of Staphylococcus, Streptococcus, Bacillus, Mycobacterium, Enterococcus, Lactobacillus, Leuconostoc, Pediococcus, and Propionibacterium. Non-limiting examples of specific species include Enterococcus faecium and Enterococcus gallinarium. Non-limiting examples of Gram-negative bacteria include bacteria found in the genera Pseudomonas, Zymomonas, Spirochaeta, Methylosinus, Pantoea, Acetobacter, Gluconobacter, Escherichia and Erwinia.

In one embodiment, the bacteria are Clostridium species, including but not limited to, Clostridium saccharobutylicum, Clostridium acetobutylicum, Clostridium beijerinckii, Clostridium puniceum, and environmental isolates of Clostridium.

Further examples of species of Clostridium contemplated for use in this invention can be selected from C. aurantibutyricum, C. butyricum, C. cellulolyticum, C. phytofermentans, C. saccharolyticum, C. saccharoperbutylacetonicum, C. tetanomorphum, C. thermobutyricum, C. thermocellum, C. puniceum, C. thermosaccharolyticum, and C. pasterianum.

Other bacteria contemplated for use in the processes and systems herein include Corynebacteria, such as C. diphtheriae, Pneumococci, such as Diplococcus pneumoniae, Streptococci, such as S. pyogenes and S. salivarus, Staphylococci, such as S. aureus and S. albus, Myoviridae, Siphoviridae, Aerobic Spore-forming Bacilli, Bacilli, such as B. anthracis, B. subtilis, B. megaterium, B. cereus, Butyrivibrio fibrisolvens, Anaerobic Spore-forming Bacilli, Mycobacteria, such as M. tuberculosis hominis, M. bovis, M. avium , M. paratuberculosis, Actinomycetes (fungus-like bacteria), such as, A. israelii, A. bovis, A. naeslundii, Nocardia asteroides, Nocardia brasiliensis, the Spirochetes, Treponema pallidium, Treponema pertenue, Treponema carateum, Borrelia recurrentis, Leptospira icterohemorrhagiae, Leptospira canicola, Spirillum minus, Streptobacillus moniliformis, Trypanosomas, Mycoplasmas, Mycoplasma pneumoniae, Listeria monocytogenes, Erysipelothrix rhusiopathiae, Streptobacillus monilformis, Donvania granulomatis, Bartonella bacilliformis, Rickettsiae, e.g., Rickettsia prowazekii, Rickettsia mooseri, Rickettsia rickettsiae, and Rickettsia conori. Other suitable bacteria may include Escherichia coli, Zymomonas mobilis, Erwinia chrysanthemi, and Klebsiella planticola.

In some embodiments, the microorganisms comprise the genera Clostridium, Enterococcus, Klebsiella, Lactobacillus, or Bacillus. In some embodiments, the microorganisms comprise Clostridium acetobutylicum, Clostridium beijerinckii, Clostridium puniceum, Clostridium saccharobutylicum, Enterococcus faecium, Enterococcus gallinarium, Clostridium aurantibutyricum, Clostridium aurantibutyricum, Clostridium tetanomorphum, or Clostridium thermosaccharolyticum.

In some embodiments, the microorganisms are obligate anaerobes. Non-limiting examples of obligate anaerobes include Butyrivibrio fibrosolvens and Clostridium species.

In other embodiments, the microorganisms are microaerotolerant and are capable of surviving in the presence of small concentrations of oxygen. In some embodiments, microaerobic conditions include, but are not limited, to fermentation conditions produced by sparging a liquid media with a gas of at least about 0.01% to at least 5% or more O₂ (e.g., 0.01%, 0.05%, 0.10%, 0.50%, 0.60%, 0.70%, 0.80%, 1.00%, 1.20%, 1.50%, 1.75%, 2.0%, 3%, 4%, 5% or more O₂). In another aspect, the microaerobic conditions include, but are not limited to, culture conditions with at least about 0.05 ppm dissolved O₂ or more (e.g., 0.05, 0.075, 0.1, 0.15, 0.2, 0.3, 0.4, 0.5, 0.6, 0.8, 1.0, 2.0, 3.0, 4.0, 5.0, 8.0, 10.0, ppm or more).

Alternatively, parent strains can be isolated from environmental samples such as wastewater sludge from wastewater treatment facilities including municipal facilities and those at chemical or petrochemical plants. The latter are especially attractive as the isolated microorganisms can be expected to have evolved over the course of numerous generations in the presence of high product concentrations and thereby have already attained a level of desired product tolerance that may be further improved upon.

Parent strains may also be isolated from locations of natural degradation of naturally occurring feedstocks and compounds (e.g., a woodpile, a saw yard, under fallen trees, landfills). Such isolates may be advantageous since the isolated microorganisms may have evolved over time in the presence of the feedstock and thereby have already attained some level of conversion and tolerance to these materials that may be further improved upon.

Individual species or mixed populations of species can be isolated from environmental samples. Isolates, including microbial consortiums can be collected from numerous environmental niches including soil, rivers, lakes, sediments, estuaries, marshes, industrial facilities, etc. In some embodiments, the microbial consortiums are strict anaerobes. In other embodiments, the microbial consortiums are obligate anaerobes. In some embodiments, the microbial consortiums are facultative anaerobes. In still other embodiments, the microbial consortiums do not contain species of Enterococcus or Lactobacillus.

When mixed populations of specific species or genera are used, a selective growth inhibitor for undesired species or genera can be used to prevent or suppress the growth of these undesired microorganisms.

### Culture Conditions

Optimal culture conditions for various industrially important microorganisms are known in the art. As required, the culture conditions may be anaerobic, microaerotolerant, or aerobic. Aerobic conditions are those that contain oxygen dissolved in the media such that an aerobic culture would not be able to discern a difference in oxygen transfer with the additional dissolved oxygen, and microaerotolerant conditions are those where some dissolved oxygen is present at a level below that found in air or air saturated solutions and frequently below the detection limit of standard dissolved oxygen probes, e.g., less than 1 ppm. The cultures can be agitated or left undisturbed. Typically, the pH of the media changes over time as the microorganisms grow in number, consume feedstock and excrete organic acids. The pH of the media can be modulated by the addition of buffering compounds to the initial fermentation media in the bioreactor or by the active addition of acid or base to the growing culture to keep the pH in a desired range. Growth of the culture may be monitored by measuring the optical density, typically at a wavelength of 600 nm, or by other methods known in the art.

Clostridium fermentations are generally conducted under anaerobic conditions. For example, ABE fermentations by C. acetobutylicum are typically conducted under anaerobic conditions at a temperature in the range of about 25° C. to about 40° C. Historically, suspension cultures did not use agitators, but relied on evolved or sparged gas to mix the contents of the bioreactors. Cultures, however, can be agitated to ensure more uniform mixing of the contents of the bioreactor. For immobilized cultures, a bioreactor may be run without agitation in a fixed bed (plug flow) or fluidized/expanded bed (well-mixed) mode. Thermophilic bacterial fermentations can reach temperatures in the range of about 50° C. to about 80° C. In some embodiments, the temperature range is about 55° to about 70° C. In some embodiments, the temperature range is about 60° C. to about 65° C. For example, Clostridium species such as C. thermocellum or C. thermohydrosulfuricum may be grown at about 60° C. to about 65° C. The pH of the Clostridium growth medium can be modulated by the addition of buffering compounds to the initial fermentation media in the bioreactor or by the active addition of acid or base to the growing culture to keep the pH in a desired range. For example, a pH in the range of about 3.5 to about 7.5, or about 5 to about 7, may be maintained in the medium for growth of Clostridium.

### Immobilization of Microorganisms on Solid or Semi-Solid Support

In some embodiment, microorganisms are grown immobilized on a solid or semi-solid support for production of one or more bioproduct(s) of interest.

Immobilization of the microorganism, from spores or vegetative cells, can be by any known method. In one embodiment, entrapment or inclusion in the support is achieved by polymerizing or solidifying a spore or vegetative cell containing solution. Useful polymerizable or solidifiable solutions include, but are not limited to, alginate, κ-carrageenan, chitosan, polyacrylamide, polyacrylamide-hydrazide, agarose, polypropylene, polyethylene glycol, dimethyl acrylate, polystyrene divinyl benzene, polyvinyl benzene, polyvinyl alcohol, epoxy carrier, cellulose, cellulose acetate, photocrosslinkable resin, prepolymers, urethane, and gelatin.

In another embodiment, the microorganisms are incubated in growth medium with a support. Useful supports include, but are not limited to, bone char, cork, clay, resin, sand, porous alumina beads, porous brick, porous silica, celite (diatomaceous earth), polypropylene, polyester fiber, ceramic, (e.g., porous ceramic, such as porous silica/alumina composite), lava rock, vermiculite, ion exchange resin, coke, natural porous stone, macroporous sintered glass, steel, zeolite, engineered thermal plastic, concrete, glass beads, Teflon, polyetheretherketone, polyethylene, wood chips, sawdust, cellulose fiber (pulp), or other natural, engineered, or manufactured products. The microorganisms may adhere to the support and form an aggregate, e.g., a biofilm.

In another embodiment, the microorganism is covalently coupled to a support using chemical agents like glutaraldehyde, o-dianisidine (U.S. Pat. No. 3,983,000), polymeric isocyanates (U.S. Pat. No. 4,071,409), silanes (U.S. Pat. Nos. 3,519,538 and 3,652,761), hydroxyethyl acrylate, transition metal-activated supports, cyanuric chloride, sodium periodate, toluene, or the like. See also U.S. Pat. Nos. 3,930,951 and 3,933,589.

In one embodiment, immobilized spores, such as those of Clostridium, e.g., C. acetobutylicum, are activated by thermal shock and then incubated under appropriate conditions in a growth medium whereby vegetative growth ensues. These cells remain enclosed in or on the solid support. After the microorganisms reach a suitable density and physiological state, culture conditions can be changed for bioproduct production. If the immobilized cells lose or exhibit reduced bioproduct production ability, they can be reactivated by first allowing the cells to sporulate before repeating the thermal shock and culture sequence.

Vegetative cells can be immobilized in different phases of their growth. For microorganisms that display a biphasic culture, such as C. acetobutylicum with its acidogenic and solventogenic phases, cells can be immobilized after they enter the desired culture phase in order to maximize production of the desired products, where in the case of C. acetobutylicum it is the organic acids acetic acid and butyric acid in the acidogenic phase and the solvents acetone, butanol and ethanol in the solventogenic phase. Alternatively, biphasic cells can be immobilized in the acidogenic phase and then adapted for solvent production.

In some embodiments, microorganisms to be immobilized in a bioreactor are introduced by way of a cell suspension. Generally, these microorganisms are dispersed in the media as single cells or small aggregates of cells. In other embodiments, the microorganisms are introduced into the bioreactor through the use of suspended particles that are colonized by the microorganisms. These suspended particles can be absorbed onto the solid support and frequently are of sufficiently small size that they can enter and become immobilized in the pore structures of the solid support. Typically, regardless of the suspended particle size, microorganisms can be transferred by contact with the solid support. A biofilm on the introduced particles can transfer to and colonize these new surfaces. In some embodiments, the desired characteristics of the microorganisms can only be maintained by culturing on a solid support, thereby necessitating the use of small colonized particle suspensions for seeding a solid support in a bioreactor.

### Support for Immobilized Microbial Growth

In some embodiments, a bioproduct producing microorganism is grown in an immobilized form on a solid or semi-solid support material in a bioreactor as described herein. In some embodiments, the support contains a porous material. Non-limiting examples of suitable support materials include bone char, synthetic polymers, natural polymers, inorganic materials, and organic materials.

Natural polymers include organic materials such as cellulose, lignocellulose, hemicellulose, and starch. Organic materials include feedstock such as plant residue and paper. Composites of two or more materials may also be used such as mixtures of synthetic polymer with natural plant polymer.

Examples of semi-solid media include alginate, κ-carrageenan and chitosan, polyacrylamide, polyacrylamide-hydrazide, agarose, polypropylene, polyethylene glycol, dimethyl acrylate, polystyrene divinyl benzene, polyvinyl benzene, polyvinyl alcohol, epoxy carrier, cellulose, cellulose acetate, photocrosslinkable resin, prepolymers, urethane, and gelatin. Examples of solid support include cork, clay, resin, sand, porous alumina beads, porous brick, porous silica, celite, wood chips or activated charcoal.

Suitable inorganic solid support materials include inorganic materials with available surface hydroxy or oxide groups. Such materials can be classified in terms of chemical composition as siliceous or nonsiliceous metal oxides. Siliceous supports include, inter alia, glass, colloidal silica, wollastonite, cordierite, dried silica gel, bentonite, and the like. Representative nonsiliceous metal oxides include alumina, hydroxy apatite, and nickel oxide.

In some embodiments, the support material is selected from bone char, polypropylene, steel, diataomaceous earth, zeolite, ceramic, (e.g., porous ceramic, such as porous silica/alumina composite), engineered thermal plastic, clay brick, concrete, lava rock, wood chips, polyester fiber, glass beads, Teflon, polyetheretherketone, polyethylene, vermiculite, ion exchange resin, cork, resin, sand, porous alumina beads, coke, natural porous stone, macroporous sintered glass, or a combination thereof. In one embodiment, the support material is bone char. Useful support material has a high surface area to volume ratio such that a large amount of active, productive cells can accumulate in the bioreactor. Useful supports may contain one or more macrostructured components containing one or more useful support material(s) that promotes good fluidmechanical properties, for example, a wire mesh/gauze packing material used for traditional distillation tower packing.

In some embodiments, the support material includes a surface area of at least about 100 m²/m³. In some embodiments, the support material comprises a bulk density of at least about 0.15 g/cm³. In some embodiments, the support material comprises a ball-pan hardness number of at least about 60. In some embodiments, the support material comprises a yield strength of at least about 20 MPa.

The particle size for the support material will vary depending upon bioreactor configuration and operation parameters. In some embodiments, the support material is sized by sieving. In some embodiments, the particles are classified by the sieve number of the mesh that they can pass through. In some embodiments, the particles are sieved with a mesh that has a U.S. Sieve Number of 3 ½, 4, 5, 6, 7, 8, 10, 12, 14, 16, 18, 20, 25, 30, 35, 40, 45, 50, 60, or 70. In some embodiments, the particles are sieved at least twice, first using a mesh with larger openings followed by a mesh with smaller openings to yield particles within a defined particle size distribution range. In some embodiments, the particles are at least about 100 μm, 200 μm, 300 μm, 400 μm, 500 μm, 600 μm, 700 μm, 800 μm, 900 μm, 1000 μm, 1,100 μm, 1,200 μm, 1,300 μm, 1,400 μm, 1,500 μm, 1,600 μm, 1,700 μm, 1,800 μm, 1,900 μm, 2,000 μm, 3,000 μm, 4,000 μm, 5,000 μm, 6,000 μm, 7,000 μm, 8000 μm, 9,000 μm, 10,000 μm, 12,500 μm, 15,000 μm, 17,500 μm, 20,000 μm, 22,500 μm, or 25,000 μm in diameter. In some embodiments, the particles are less than about 100 μm, 200 μm, 300 μm, 400 μm, 500 μm, 600 μm, 700 μm, 800 μm, 900 μm, 1000 μm, 1,100 μm, 1,200 μm, 1,300 μm, 1,400 μm, 1,500 μm, 1,600 μm, 1,700 μm, 1,800 μm, 1,900 μm, 2,000 μm in diameter. In further embodiments, at least about 80%, 85%, 90%, 95%, or 100% of the particle have diameters that are in the range of about 100-400 μm, 100-600 μm, 100-800 μm, 200-500 μm, 200-800 μm, 200-1000 μm, 400-800 μm, 400-1000 μm, 500-1000 μm, 600-1,200 μm, 800-1,400, μm 1,000-1,500, μm 1,000-2000 μm, 2,000-4,000 μm, 4,000-6,000 μm, 5,000-12,000 μm, 3,000-15,000 μm, or 6,000-25,000 μm. In some embodiments, the particle diameters are the equivalent diameters, a parameter that takes into account the irregular shapes of the individual particles.

Ideally, the semi-solid or solid support material should have a high surface area. This can be achieved through the use of small sized particles, particles with high porosity, or a combination thereof. In some embodiments, the surface area of the particles is at least about 0.003 m²/g, 0.01 m²/g, 0.02 m²/g, 0,05 m²/g, 0.1 m²/g, 0.5 m²/g, 1 m²/g, 5 m²/g, 10 m²/g, 25 m²/g, 50 m²/g, 75 m²/g, 100 m²/g, 125 m²/g, 150 m²/g, 175 m²/g, 200 m²/g, 225 m²/g, 250 m²/g, 275 m²/g, 300 m²/g, 325 m²/g, 350 m²/g, 375 m²/g, 400 m²/g, 425 m²/g, 450 m²/g, 500 m²/g, 600 m²/g, 700 m²/g, 800 m²/g, 900 m²/g, 1000 m²/g, or 2000 m²/g. Additionally, the bulk density should be sufficiently high so that the smallest particles settle out of the fluid stream in the column expansion zone and/or particle disengagement zone and are thereby retained in the bioreactor. In some embodiments, the bulk density of the support is at least about 0.1 g/cm³, 0.2 g/cm³, 0.3 g/cm³, 0.4 g/cm3, 0.5 g/cm³, 0.6 g/cm³, 0.7 g/cm³, 0.8 g/cm³, 0.9 g/cm³, 1.0 g/cm³, 1.1 g/cm³, 1.2 g/cm³, or 1.3 g/cm³. The support material should have sufficient hardness to resist abrasion and thereby avoid appreciable dust formation when the support particles touch or collide with each other. In some embodiments, the support has a ball-pan hardness number of at least about 20, 40, 60, 80, 100, 120, 140, 160 or 200. The support material should also have sufficient tensile strength to resist shattering due to internal stresses, which may be caused by the growth of biofilms inside support material pores. In some embodiments, the support has a yield strength of at least about 20 MPa, 40 MPa, 60 MPa, 80 MPa, 100 MPa, 120 MPa, 140 MPa, 160 MPa, 180 MPa, 200 MPa, 300 MPa, or 400 MPa. The support material should also have the ability to resist being crushed by the accumulated weight of material above it. Crush strength is another measurement of the mechanical strength of the support and is typically a function of the composition, shape, size, and porosity of the material (increase in port volume may negatively impact particle strength). In some embodiments, the crush strength is at least about 8 kg.

In some embodiments, the support material is chosen to support growth of the fermenting bioproduct producing microorganism as a biofilm. The biofilm may grow on exterior surfaces of support particles, in the fluid space between support particles, and/or on surfaces in the interior of pores of the support material.

### Continuous Process

In some embodiments, a continuous process for bioproduct production is provided. In a continuous production process herein, a conditioned carbohydrate-containing feedstock containing soluble sugar molecules is continuously fed to one or more bioreactors for microbial production of the bioproduct, the bioproduct is continuously produced by immobilized microorganism(s) in the one or more bioreactors, and bioproduct-containing effluent, i.e., fermentation broth, is continuously withdrawn from the one or more reactors, for the duration of fermentation. In some embodiments, feedstock is continuously hydrolyzed to release soluble sugar molecules, and continuously conditioned prior to introduction of the conditioned hydrolyzed feedstock into the bioreactor(s). The conditioning process may operate continuously downstream from a feedstock hydrolysis process, and upstream from the bioreactor(s), and conditioned hydrolyzed feedstock may be continuously fed to the bioreactor for the duration of fermentation. In one embodiment, the feedstock is lignocellulosic feedstock, and is hydrolyzed with nitric acid to release soluble sugar molecules from cellulose and hemicellulose, as described supra.

In some embodiments, the continuous process may also include downstream continuous concentration and/or purification processes for recovery of the bioproduct, wherein continuously withdrawn effluent is continuously processed in one or more concentration and/or purification processes to produce a bioproduct.

In some embodiments, the process may also include deconstruction of the feedstock and/or removal of extractives from the feedstock, as described herein. Deconstruction and/or removal of extractives may be continuous or may occur prior to or periodically throughout the continuous process.

In some embodiments, the process operates continuously for at least about 50, 100, 200, 300, 400, 600, 800, 1000, 1350, 1600, 2000, 2500, 3000, 4000, 5000, 6000, 7000, 8000, or 8400 hours.

A “continuous” process as described herein may include periodic or intermittent partial or complete shutdowns of one or more parts of the bioproduct production system for processes such as maintenance, repair, regeneration of resin, etc.

Continuous fermentation, with constant feed of feedstock and withdrawal of product-containing microbial broth, can minimize the unproductive portions of a fermentation cycle, such as lag, growth, and turnaround time, thereby reducing the capital cost, and can reduce the number of inoculation events, thus minimizing operational costs and risk associated with human and process error.

The continuous methods and systems described herein can utilize one or more, e.g., one, two, or three or more, bioreactors. When multiple (two or more) bioreactors are used, they may be arranged in parallel, series, or a combination thereof. The bioreactors can grow the same or different strains of microorganism(s). The strains can be different based on the type of sugar they metabolize to maximize bioproduct production. For example, a first bioreactor or multiple bioreactors arranged in parallel, series, or a combination thereof can grow a strain that has been selected to metabolize C5 sugars and a second bioreactor or multiple bioreactors arranged in parallel, series, or a combination thereof can grow another strain that has been selected to metabolize C5 and C6 sugars. The bioreactors may be coupled to upstream feedstock hydrolysis and conditioning units, and may also be coupled to a downstream recovery/separation unit. For example, an acid hydrolysis unit may be coupled to an in fluid communication with a downstream conditioning unit, for conditioning with a metal salt as described herein, which is coupled to a downstream bioreactor, whereby conditioned hydrolysate, which has been separated from metal salt complexed inhibitor(s) is fed to the downstream bioreactor for production of bioproduct(s) of interest. Hydrolysis, conditioning, product production, and product recovery may proceed continuously.

In one embodiment in which lignocellulosic material is hydrolyzed by nitric acid in two stages, as described above, a first bioreactor or multiple bioreactors arranged in parallel, series, or a combination thereof with a strain that metabolizes C5 sugars can be coupled to an upstream first stage hydrolysis module of a nitric acid hydrolysis unit for hydrolysis of lignocellulosic feedstock and an upstream conditioning unit downstream from the hydrolysis unit and upstream from the bioreactor(s). A second bioreactor or multiple bioreactors arranged in parallel, series, or a combination thereof with a strain that metabolizes C5 and C6 sugars can be coupled to an upstream second stage hydrolysis module of a nitric acid hydrolysis unit for hydrolysis of a lignocellulosic feedstock and an upstream conditioning unit downstream from the hydrolysis unit and upstream from the bioreactor(s). Alternatively, the same bioreactor or multiple bioreactors arranged in parallel, series, or a combination thereof may be used for conversion of both C5 and C6 sugars to bioproduct. For example, both first and second stage conditioned nitric acid hydrolysates of a lignocellulosic feedstock may be added either separately or as a combined mixture to the bioreactor(s).

The following examples are intended to illustrate, but not limit, the invention.

### EXAMPLES

### Example 1

A hydrolysate was prepared from beetle killed Lodgepole pine using nitric acid as the catalyst for the hydrolysis reaction. The following conditions were used for hydrolysis: nitric acid concentration 0.4-0.5% on a dry wood basis, pH approximately 1.9-2.2, temperature 170° C., time 7 minutes, approximately 25-30% solids in the feed.

The raw hydrolysate was measured out into 100 ml glass bottles to volumes of 50 ml using a pipette. The pH of the hydrolysate samples was then adjusted to pH values in the range of 5.5 to 10 with a 15% solution of ammonium hydroxide.

Aluminum sulfate and ferric chloride were added at concentrations in the range of 2 g/L to 5 g/L, with pH ranging from 9 to 10.5, and the solutions incubated for about 30 minutes at temperatures in the range of 20° C. to 40° C. The solutions were mixed during the incubation using a magnetic stir plate. The solutions were then filtered through a 0.2 micron filter to separate precipitate from the liquid hydrolysate.

The solutions were then cooled to room temperature if not already at room temperature. The pH of the solutions was then adjusted to 7.2 with nitric acid or ammonium hydroxide. After pH adjustment, 10 ml of each solution was then filtered through a Pall sterile syringe filter with a pore size of 0.2 microns into a 15 ml falcon tube. The solutions were then placed in an anaerobic hood overnight to de-oxygenate.

Media components were added to the de-oxygenated hydrolysate solution at the prescribed concentrations to support microbial growth (i.e., growth media components and trace elements). The tubes were then inoculated with a butanol-producing Clostridium strain at a concentration of approximately 5×10⁷ CFU. The conditions used for fermentation were as follows: volume 10 ml, pH approximately 6.8 before inoculation, temperature 30° C.

Aluminum sulfate and ferric chloride were both successful in transforming an otherwise un- fermentable hydrolysate into a fermentable feedstock that supported microbial growth and production of butanol. Under the conditions used for fermentation, aluminum sulfate produced a feedstock that resulted in higher butanol production along with less precipitate in the final product than ferric chloride. The best results for treatment of raw hydrolysate with aluminum sulfate and ferric chloride were at the following conditions: metal salt concentration 3 g/L aluminum sulfate or 2 g/l ferric chloride, pH 9.5, room temperature (about 20° C.). The butanol concentrations after microbial fermentation for 72 hours were 8.64 g/L and 7.69 g/L for aluminum sulfate and ferric chloride, respectively.

Adjustment of hydrolysate pH before metal salt addition was found to be important. For example, one hydrolysate composition adjusted to pH 9 before addition of metal salts did not ultimately support microbial growth. However, adjustment of the solution to pH 9.5 resulted in a conditioned hydrolysate in which the microorganism grew and produced butanol.

Lower temperatures also resulted in lower sugar loss. At room temperature, the sugar loss was less than 15%.

### Example 2

Formic acid is toxic to Clostridium species. Formic acid is produced during fermentation and builds to toxic levels. Formic acid is also present in hydrolysates of lignocellulosic biomass. Formic acid was determined to become inhibitory to growth of Clostridium and the subsequent production of butanol at concentrations of 0.3 to 0.4 g/l in the fermentation medium. Aluminum sulfate precipitation was investigated as an effective means for removal of formic acid from biomass hydrolysates, thereby reducing the toxicity of the hydrolysate to improve butanol production. Aluminum sulfate precipitation reduced the concentration of formic acid in raw hydrolysate to a non-toxic level.

A nitric acid hydrolysate was prepared from beetle killed Lodgepole pine as described in Example 1. The pH of the hydrolysate samples was then adjusted to pH 10 with a 15% solution of ammonium hydroxide. Aluminum sulfate was added at a concentration of 3 g/L, pH 9.5, and the solution was incubated for about 30 minutes at 20° C. The solution was mixed and filtered as described in Example 1.

The solution was cooled to room temperature, pH adjusted and sterile filtered as described in Example 1. Formic acid concentration was 0.27 g/L in the conditioned hydrolysate, in comparison to 1.31 g/L in untreated hydrolysate. Sugar loss was about 14.5%.

The solution was then placed in an anaerobic hood overnight to de-oxygenate. Media components were added to the de-oxygenated hydrolysate solution at the prescribed concentrations to support microbial growth (i.e., growth media components and trace elements).

The growth media was then inoculated with a butanol-producing Clostridium strain at a concentration of approximately 5×10⁷ CFU, and fermented as described in Example 1. The butanol concentration after microbial fermentation for 72 hours with aluminum sulfate treated hydrolysate was 8.64 g/L, with a yield of 0.36, in comparison with no discernable butanol production for untreated hydrolysate.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated in the appended claims.

All publications, patents, and patent applications cited herein are hereby incorporated by reference in their entireties for all purposes and to the same extent as if each individual publication, patent, or patent application were specifically and individually indicated to be so incorporated by reference.

We claim:

1. A method for removing at least a portion of at least one inhibitor of microbial growth and/or microbial bioproduct production from a composition that comprises said at least one inhibitor and at least one compound suitable for use as a carbon source in microbial fermentation, said method comprising:
contacting the composition with a metal salt that comprises a metal with a valence of three or greater or a divalent metal that does not comprise calcium, wherein the metal salt forms a complex with said at least one inhibitor in the composition; and
separating the complex from the composition to form a conditioned composition.

2. A method according to claim 1, wherein the salt comprises a metal selected from aluminum, or iron, or magnesium.

3. A method according to claim 2, wherein the salt comprises a trivalent metal.

4. A method according to claim 3, wherein the salt comprises aluminum, iron, or a combination thereof.

5. A method according to claim 4, wherein the metal salt is Al₂(SO₄)₃ or FeCl₃.

6. A method according to claim 1, wherein the pH of the composition is adjusted to about 7 to about 11.

7. A method according to claim 1, wherein the method is performed at a temperature of about 20° C. to about 60° C.

8. A method according to claim 1, wherein the composition is a hydrolysate of cellulosic biomass.

9. A method according to claim 8, wherein the cellulosic biomass comprises a lignocellulosic biomass.

10. A method according to claim 9, wherein the lignocellulosic biomass comprises softwood or hardwood, or a combination thereof.

11. A method according to claim 8, wherein the cellulosic biomass comprises grass or straw, or a combination thereof.

12. A method according to claim 8, wherein the cellulosic biomass comprises bagasse, cane trash, seaweed, algae, microalgae, agricultural waste or residue, hyacinth, sorghum, sugar beets, soybean residue, palm oil residue, or pulp mill liquor or effluent.

13. A method according to claim 8, wherein said hydrolysate is produced by acid hydrolysis of said cellulosic biomass.

14. A method according to claim 15, wherein said acid comprises nitric acid, formic acid, acetic acid, phosphoric acid, hydrochloric acid, sulfuric acid, or a combination thereof.

15. A method according to claim 14, wherein said acid hydrolysis comprises hot water extraction of said biomass.

16. A method according to claim 15, further comprising addition of acid after said hot water extraction to further hydrolyze carbohydrate polymers.

17. A method according to claim 1, wherein the composition comprises glycerol, dairy effluent, or a hydrolysate of food waste.

18. A method according to claim 1, wherein the complex is separated from the composition by filtration, centrifugation, pressing, or decantation.

19. A method according to claim 1, wherein the at least one inhibitor comprises an organic acid, an aldehyde, a lignin, a lignin derivative, an inorganic salt, a fatty acid, a fatty alcohol, a fat, a wax, a polyester, a terpenoid, an alkane, a wood extractive, and a Hibbert's ketone.

20. A method according to claim 21, wherein the at least one inhibitor comprises formic acid.

21. A method according to claim 1, wherein the composition comprises sugar molecules, and wherein less than about 15% of the sugar molecules are degraded in said method.

22. A conditioned composition produced according to the method of claim 1.

23. A method for producing a bioproduct, comprising culturing a microorganism in a medium comprising a conditioned composition according to claim 22, wherein the growth of and/or bioproduct production in the microorganism is greater in the medium comprising the conditioned composition in comparison to a medium that comprises an identical composition that has not been conditioned.

24. A method according claim 23, wherein the bioproduct is a biofuel selected from butanol, ethanol, and acetone, or a combination thereof.

25. A method for producing a bioproduct, comprising culturing a microorganism in a medium comprising a conditioned composition according to claim 22, wherein the bioproduct is produced at a greater titer, yield, and/or productivity in the medium comprising the conditioned composition in comparison to a medium that comprises an identical composition that has not been conditioned.
